(19) United States
(12) Patent Application Publication
(10) Pub. No.: US 2007/0214509 A1
(43) Pub. Date: Sep. 13, 2007

(54) Methods and systems for identifying compounds that modulate alpha-synuclein aggregation

(75) Inventor: J. William Langston, Sunnyvale, CA (US)

Correspondence Address: BUCHANAN, INGERSOLL & ROONEY LLP, P.O. BOX 1404, ALEXANDRIA, VA, 22313-1404, US

(73) Assignee: The Parkinson's Institute, Sunnyvale, CA (US)

(21) Appl. No.: 11/644,616

(22) Filed: Dec. 22, 2006

Related U.S. Application Data
(60) Provisional application No. 60/753,538, filed on Dec. 23, 2005.

(51) Int. Cl. A01K 67/033; C12Q 1/68; G01N 33/567

(52) U.S. Cl. 800/3; 435/6; 435/7.2

(57) ABSTRACT

This invention relates to the field of chemical sensing and signal transduction using devices that are sensitive to single molecular species or closely related families of species. In one aspect, the present invention relates to systems that are designed to detect target molecules in the gas phase, as well as more condensed phases, in the presence of heterogeneous populations of molecular species.

### CROSS REFERENCE TO RELATED APPLICATIONS

This application claims priority to U.S. Provisional Application Ser. No. 60/753,538 filed Dec. 23, 2005, the disclosure of which is incorporated herein by reference.

### TECHNICAL FIELD

Methods for identifying compounds that modulate synuclein aggregation are provided. Also provided are systems that include two or more methods for identifying compounds that modulate synuclein aggregation.

### BACKGROUND

Alpha-synuclein is one of hundreds of proteins in human brain cells. It appears in many cells throughout the body as a tubular structure under normal conditions. The tube folds naturally, but can be influenced to fold incorrectly, creating fibrils. Fibrils can aggregate and are either too dense or too large to be flushed from the cell by proteosomes, which are designed for just such cell cleansing. As the alpha-synuclein protein aggregates, it can collect additional proteins and eventually turn into a “Lewy body.” Lewy bodies are involved in the etiology of a variety of neurologic disorders, including Parkinson's Disease, Parkinson's Disease with accompanying dementia, Lewy body dementia, Alzheimer's disease with Parkinsonism, and multiple system atrophy.

Aggregation of alpha-synuclein may be due to over-expression and accumulation of the protein. Alternatively or concurrently, the aberrant shape or conformation of some molecules of alpha-synuclein may be impressed upon other synuclein molecules. These molecules then bind to one another and the protein aggregates accumulate and deposit inside the neuron, where they exert oxidative damage as they increase in size. This process first prevents the neuron from performing its necessary role in brain function and as it progresses, eventually kills the neuron.

Parkinson's Disease has a prevalence of about 2% after age 65, and, thus, is one of the most common neurodegenerative human disorders. Its pathological hallmarks are: (a) the presence of Lewy bodies (Spillantini, et al., 1997; Nature 388:839-40), round cytoplasmic inclusions about 5-25 μm in diameter, mainly reactive for alpha-synuclein but also for ubiquitin and other proteins; and (b) massive loss of dopaminergic neurons in the pars compacta of the substantia nigra (Fearnley, et al., 1991; Brain 114:2283).

Effective treatments for neurodegenerative diseases such as Parkinson's Disease are needed. Accordingly, a need exists for rapidly screening compounds to identify those that specifically prevent or inhibit the pathological aggregation of alpha-synuclein and/or disperse the toxic aggregates.

### SUMMARY

Provided herein are methods and systems for identifying agents that modulate alpha-synuclein aggregation. The methods of the present invention are useful to determine the anti-aggregation potential of agents or to screen for agents with anti-aggregation or dis-aggregating properties.

In one embodiment, a method for identifying an agent that modulates alpha-synuclein aggregation is provided. The methods includes providing a plurality of candidate agents and 1) contacting each candidate agent with a polypeptide comprising alpha-synuclein and 2) determining whether the agent promotes alpha-synuclein dis-aggregation, or inhibits alpha-synuclein aggregation, in vitro. The method further includes administering an agent identified in the in vitro assay to a non-mammalian organism comprising neuronal cells that over-express a polypeptide comprising alpha-synuclein and determining whether the agent promotes alpha-synuclein dis-aggregation, or inhibits alpha-synuclein aggregation, by detecting a change in the aggregation of the over-expressed alpha-synuclein. The method also includes administering an agent identified in the non-mammalian in vivo assay to a transgenic mammalian organism comprising neuronal cells that over-express a polypeptide comprising alpha-synuclein in vivo and determining whether the agent promotes alpha-synuclein dis-aggregation, or inhibits alpha-synuclein aggregation, by detecting a change in the aggregation of the over-expressed alpha-synuclein. The method optionally includes administering an agent identified in the transgenic non-human mammalian assay to a non-human primate genetically modified to over-express a polypeptide comprising alpha-synuclein in vivo and determining whether the agent promotes alpha-synuclein dis-aggregation, or inhibits alpha-synuclein aggregation by detecting a change in the aggregation of the over-expressed alpha-synuclein.

In one embodiment of the invention, the neuronal cells are dopaminergic cells. In some aspects, the neuronal cells are genetically modified to over-express alpha-synuclein. In some embodiments, the alpha-synuclein is human alpha-synuclein. In other embodiments, the over-expressed alpha-synuclein is mutant alpha-synuclein. In some aspects, the mutant alpha-synuclein is mutant human alpha-synuclein A53T.

In another embodiment, the organism used in methods provided herein is Caenorhabditis elegans (C. elegans). In other embodiments, the organism is a murine organism.

In some aspects of the invention, the detection of alpha-synuclein aggregation is by microscopy. In other aspects, the detection is by immunofluorescence.

In one embodiment, agents identified by methods of the invention are suitable for treating a condition associated with alpha-synuclein aggregation.

In other embodiments, the agent is selected from the group consisting of a chemical, a therapeutic molecule, a biomolecule, and a virus. The chemical may be a small molecule. The therapeutic molecule may be any therapeutic molecule, such as an antibiotic. Moreover, the biomolecule may be a polypeptide. In some aspects, the polypeptide is a peptoid. In other aspects the biomolecule is a nucleic acid such as DNA or RNA. In some aspects, the RNA may be anti-sense RNA. In other aspects, the RNA is siRNA.

In another embodiment, a method of treating an individual suffering from a disease associated with alpha-synuclein aggregation is provided. The method includes administering to the individual a pharmaceutical composition comprising a therapeutically effective amount of an agent identified by a method or system provided herein.

In another embodiment, a method of treating an individual suffering from Parkinson's Disease is provided. The method includes administering to the individual a pharmaceutical composition comprising a therapeutically effective amount of an agent identified by a method or system provided herein.

In another embodiment, a system for identifying an agent that modulates alpha-synuclein aggregation is provided. The system includes practicing a method provided herein and communicating the results of the method to a database. The results may be accessed by multiple users. In some aspects, the information is optionally correlated with information contained in other databases.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 is a flow diagram depicting an embodiment of the invention for screening agents that modulate alpha-synuclein aggregation.

### DETAILED DESCRIPTION

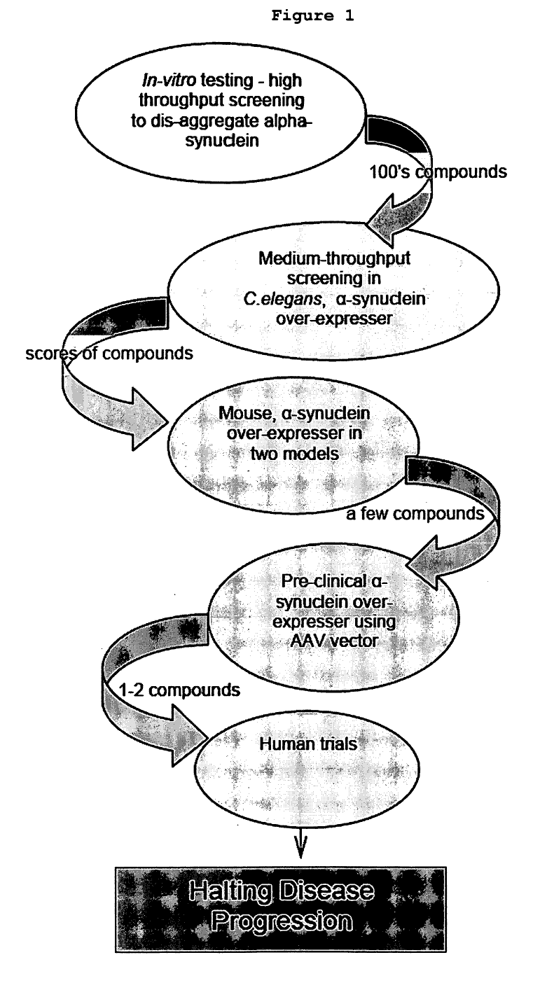

Described herein are methods and systems for identifying compounds modulate the aggregation of alpha-synuclein. Referring to FIG. 1, a library containing a plurality of candidate agents may be evaluated to determine the most desirable subsets, or “best-in-class” members. Such libraries can be generated on the basis of their expected binding affinities to alpha-synuclein, and derivatives thereof. The most desirable subsets (i.e., lead candidate agents) are then screened in subsequent assays to identify those that display optimal alpha-synuclein modulating activity. In this manner, successive rounds of screening can be used to identify the agent that effectively modulates alpha-synuclein activity.

“Modulation of alpha-synuclein aggregation” includes prevention, inhibition or reversal of aggregation. Thus, “modulation” includes promoting the dis-aggregation of alpha-synuclein aggregates. The term “alpha-synuclein” includes mutant and wild-type polypeptides, and fragments thereof provided that such fragments are capable of participating in the formation of aggregates. An “activity” or “function” of alpha-synuclein includes, but is not limited to, formation of inclusions/aggregation in the cytoplasm, association with cell membrane, interaction with an alpha-synuclein associated protein. In addition, alpha-synuclein can inhibit phospholipase D (PLD) activity, cause toxicity to cells, and lead to impaired proteasomal activity. For example, the identified agent may prevent alpha-synuclein misfolding, inhibit formation of alpha-synuclein inclusions/aggregation, or promote alpha-synuclein disaggregation. Accordingly, irrespective of the exact mechanism of action, agents identified by the screening methods described herein will provide therapeutic benefit to alpha-synuclein associated diseases.

FIG. 1 provides a general description of methods and systems used to screen and identify compounds that modulate alpha-synuclein aggregation. Initially, the ability of various agents to modulate alpha-synuclein aggregation in vitro will be tested. Since alpha-synuclein can be grown and aggregated in a cell culture, an exemplary assay may include contacting aggregated alpha-synuclein with a candidate agent and determining whether the agent promotes dis-aggregation of alpha-synuclein. Exemplary agents shown to have such an effect include rifampin, rifampicin, rifamycin, or rifaldazine. It is understood that alpha-synuclein aggregation may be attained in the presence or absence of a cellular environment. For example, international application WO00/20020 (Masliah; published Apr. 13, 2000) describes methods of screening alpha synuclein anti-aggregating compounds using metal induced alpha synuclein aggregation and Thioflavin-S staining.

Accordingly, certain aspects of the present disclosure provide methods (assays) of screening for a candidate agent (drug or compound) and identifying an agent for treating a disease associated with alpha-synuclein aggregation. A “candidate agent” as used herein, is any substance with a potential to reduce, interfere with or block alpha-synuclein aggregation. Aggregation may be attributable to over-expression of alpha-synuclein or expression of abnormally processed alpha-synuclein. Various types of candidate agents may be screened by the methods described herein, including nucleic acids, polypeptides, small molecule compounds, and peptidomimetics.

In one embodiment, initial screening methods described herein may include any assay that provides for the identification of agents that prevent alpha-synuclein misfolding, inhibit formation of alpha-synuclein inclusions/aggregation, or promote alpha-synuclein disaggregation. Exemplary assays include the use of yeast cells, bacterial cells or animal cells that are engineered to express, or over-express, an alpha-synuclein polypeptide

Candidate agents include chemicals (including polymers, organic compounds, etc.); therapeutic molecules (including therapeutic drugs, antibiotics, etc.); biomolecules (including hormones, cytokines, proteins, lipids, carbohydrates, cellular membrane antigens); receptors (neural, hormonal, nutrient, and cell surface receptors) or their ligands; and viruses (including retroviruses, herpesviruses, adenoviruses, lentiviruses, etc.).

Candidate agents may be screened from large libraries of synthetic or natural compounds. One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can immediately be used in the screens.

Candidate agents may include a small molecule. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In other embodiments, candidate agents also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulphydryl or carboxyl group.

Other suitable candidate agents may include antisense molecules, ribozymes, and antibodies (including single chain antibodies), each of which would be specific for the target molecule. For example, an antisense molecule that binds to a translational or transcriptional start site, or splice junctions, would be ideal candidate inhibitors.

One embodiment contemplates screening assays using fluorescence activated cell sorting (FACS) analysis. FACS is a technique well known in the art, and provides the means of scanning individual cells for the presence of fluorescently labeled/tagged moiety. The method is unique in its ability to provide a rapid, reliable, quantitative, and multiparameter analysis on either living or fixed cells. For example, alpha-synuclein can be suitably labeled, and provide a useful tool for the analysis and quantitation of alpha-synuclein aggregation and fibril and/or aggregate formation.

In other embodiments, methods of the present disclosure relate to determining proteasomal impairment caused by alpha-synuclein. In yet other embodiments, methods of the present disclosure relate to determining oxidative stress caused by alpha-synuclein. Mitochondrial dysfunction and oxidative stress are clearly linked to diseases (e.g., Parkinson's disease) but in ways still poorly understood. Certain embodiments provide methods of further testing those lead candidate agents that have been identified in the initial screening assay, in other model systems. The model systems include, but are not limited to, worms, flies, mammalian cells, and in vivo animal models (e.g., an alpha-synuclein transgenic mouse).

Accordingly, lead candidate agents identified in the in vitro screening assay may be further tested to identify those agents that promote alpha-synuclein dis-aggregation in vivo. An exemplary test includes using C. elegans (Caenorhabditis elegans) neuronal cells which have been genetically modified to over-express alpha-synuclein and to fluoresce the dopaminergic neurons. The action of any agent can be revealed in real time, through a microscope. The C. elegans nervous system has neuronal cells similar to those found in humans, but in much smaller numbers. The process may be monitored from remote locations.

Lead candidate agents identified from the C. elegans assay may then be tested in mouse neurons. Transgenic mice that over-express alpha-synuclein and display alpha-synuclein aggregation have been developed. Using these genetically altered mice, experiments can be conducted quickly and efficiently to see which agents may have the desired effect in the neuronal cells of a warm-blooded vertebrate. During this phase, the mice with excessive alpha-synuclein aggregation will be treated with the agents and then examined to identify those compounds that prevent, inhibit or reverse aggregation.

Subsequently, lead candidate agent(s) may be administered to a non-human primate to confirm the benefit of the compound. Such non-human primates may be genetically modified to over-express the alpha-synuclein, as in the mouse. Once a lead candidate agent has proved effective in non-human primates, small scale clinical trials may be commenced.

As previously noted, alpha-synuclein useful in the methods and systems of the invention includes mutant and wild-type polypeptides, and fragments thereof provided that such fragments are capable of participating in the formation of aggregates. Fragments of alpha-synuclein include, for example, the non-amyloid component (NAC) fragment that is a constituent of Alzheimer's disease amyloid plaques.

Exemplary mutants of alpha-synuclein include two different point mutations in the alpha-synuclein gene (A53T and A30P) that were identified in separate families with dominantly transmitted Parkinson's disease (see, e.g., international application WO 98/5950, published Dec. 30, 1998). These point mutations of alpha-synuclein have been shown to increase the ability of alpha synuclein to aggregate and to slow down degradation of the mutated alpha synuclein. The consistent effect of these mutations in increasing the amount and aggregation of alpha-synuclein suggests that these processes play an important role in the pathophysiology of various neurodegenerative disorders. In fact, overexpression of wild-type alpha-synuclein is associated with cellular toxicity. Thus, agents that inhibit alpha-synuclein aggregation represent a novel therapeutic strategy as disease-modifying agents for neurodegeneration.

Referring again to FIG. 1, the invention provides systems for identifying an agent that modulates alpha-synuclein aggregation. Such a system may include mechanisms for performing various parts of a method for identifying an agent that that modulates alpha-synuclein aggregation at remote locations. For example, candidate agents may be screened in an in vitro assay for activity associated with promoting alpha-synuclein dis-aggregation, or inhibiting alpha-synuclein aggregation. The in vitro screen may be performed at a first location and the results transmitted to a second or central location. The results may be accessed by multiple users such that subsequent in vivo assays may be performed using those agents identified by the in vitro assay as possessing desirable activities against alpha-synuclein aggregation. The results of the in vivo assays may similarly be transmitted to a central location for storage and access by other users. Accordingly, the information generated by multiple users at various locations can be communicated to a central location, such as a server, and compiled. The compilation of assay information can entail the construction of databases that reflect the results of the various in vitro and in vivo assays performed on the plurality of candidate agents. Thus, the invention further provides a computer system including a database incorporating records of the activity of any or all of a plurality of agents that may modulate alpha-synuclein aggregation. Such a database including results inputted by one or more users of the database; a processor for cross-assembling the results; a processor for correlating the information and for generating an index of agents that promote alpha-synuclein dis-aggregation or inhibit alpha-synuclein aggregation, and a means for outputting to an output device the results of the index.

It is therefore immediately evident that a computer program designed for storing, correlating and indexing the results of the in vitro and in vivo assays may be provided which is suitable for use with the methods and systems of the present invention. Accordingly, the invention provides methods and systems for indexing the results of various screening assays and identifying those agents that may be used in a therapeutic composition for treating a disorder associated with alpha-synuclein aggregation.

It is further envisioned that the method and systems of the present invention can be integrated with other methods of identifying agents suitable for modulating alpha-synuclein aggregation. For example, a wide variety of structural, chemical, and sequence information is available for agents that may be screened by a method or system of the invention. For example, structural information for polypeptides can be generated in silico based solely on the amino acid content of the polypeptide. Similarly, crystallographic structures of candidate polypeptides are available through various databases known to the skilled artisan. Chemical information for small molecules that may be screened by the present methods and systems are also available from various databases. In addition, sequence information for nucleic acid molecules, such as antisense RNA or siRNA (small interfering RNA), is similarly available through various databases. The integration of the information generated by a method or system of the present invention can be correlated with the information contained in such databases in order to design agents that not only modulate alpha-synuclein aggregation, but also are suitable for administration to a patient. Thus, cross-indexing the information generated in each phase of a method or system provided herein with information contained in other databases facilitates that identification of agents suitable for use as a therapeutic. For example, the information generated by the invention can be compared to other indices in order to facilitate the identification of common structures, features or sequences of agents that modulate alpha-synuclein aggregation.

Accordingly, aspects of the invention may be implemented in hardware or software, or a combination of both. The algorithms and processes of the invention may be implemented in one or more computer-programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

As previously noted, the invention provides methods and systems for identifying those agents that may be used in a therapeutic composition for treating a disorder associated with alpha-synuclein aggregation. In certain embodiments, candidate agents (i.e., drugs or compounds) may be formulated in combination with a suitable pharmaceutical carrier. Such formulations comprise a therapeutically effective amount of the agent, and a pharmaceutically acceptable carrier (excipient). Examples of suitable carriers are well known in the art. To illustrate, the pharmaceutically acceptable carrier can be an aqueous solution or physiologically acceptable buffer. Optionally, the aqueous solution is an acid buffered solution. Such acid buffered solution may comprise hydrochloric, sulfuric, tartaric, phosphoric, ascorbic, citric, fumaric, maleic, or acetic acid. Alternatively, such carriers include, but are not limited to, saline, buffered saline, dextrose, water, glycerol, ethanol, and combinations thereof. Formulations will suit the mode of administration, and are well within the skill of the art.

In certain embodiments of such methods, one or more agents can be administered, together (simultaneously) or at different times (sequentially). In addition, such agents can be administered with another type(s) of drug(s) for treating a disease associated with alpha-synuclein aggregation. For example, the identified agent may be administered together with Levodopa (L-DOPA) for treating Parkinson's disease.

The phrase “therapeutically effective amount,” as used herein, refers to an amount that is sufficient or effective to prevent or treat (prevent the progression of or reverse) a disease associated with alpha-synuclein aggregation, including alleviating symptoms of such diseases.

The dosage range depends on the choice of the agent, the route of administration, the nature of the formulation, the nature of the subject's condition, and the judgment of the attending practitioner. Wide variations in the needed dosage, however, are to be expected in view of the variety of drugs available and the differing efficiencies of various routes of administration. For example, oral administration would be expected to require higher dosages than administration by intravenous injection. Variations in these dosage levels can be adjusted using standard empirical routines for optimization, as is well understood in the art.

The examples set forth above are given to provide those of ordinary skill in the art with a complete disclosure and description of how to make and use various embodiments of the methods and systems disclosed herein, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying an agent, the method comprising:
a) providing a plurality of candidate agents;
b) contacting each candidate agent with a polypeptide comprising alpha-synuclein and determining whether the agent promotes alpha-synuclein dis-aggregation, or inhibits alpha-synuclein aggregation, in vitro;
c) administering an agent identified in b) to a non-mammalian organism comprising neuronal cells that over-express a polypeptide comprising alpha-synuclein and determining whether the agent promotes alpha-synuclein dis-aggregation, or inhibits alpha-synuclein aggregation, by detecting a change in the aggregation of the over-expressed alpha-synuclein;
d) administering an agent identified in c) to a transgenic mammalian organism comprising neuronal cells that over-express a polypeptide comprising alpha-synuclein in vivo and determining whether the agent promotes alpha-synuclein dis-aggregation, or inhibits alpha-synuclein aggregation, by detecting a change in the aggregation of the over-expressed alpha-synuclein; and
e) optionally administering an agent identified in d) to a non-human primate genetically modified to over-express a polypeptide comprising alpha-synuclein in vivo and determining whether the agent promotes alpha-synuclein dis-aggregation, or inhibits alpha-synuclein aggregation by detecting a change in the aggregation of the over-expressed alpha-synuclein.

2. The method of claim 1, part c), wherein the neuronal cells are dopaminergic cells.

3. The method of claim 1, part c), wherein the organism is Caenorhabditis elegans (C. elegans).

4. The method of claim 1, part c), wherein the detecting is by microscopy.

5. The method of claim 1, part d), wherein the neuronal cells are dopaminergic cells.

6. The method of claim 1, part d), wherein the organism is a mouse.

7. The method of claim 1, part d), wherein the neuronal cells are genetically modified to over-express alpha-synuclein.

8. The method of claim 7, wherein the alpha-synuclein is human alpha-synuclein.

9. The method of claim 1, part b), c), d) or e), wherein the over-expressed alpha-synuclein is a mutant alpha-synuclein.

10. The method of claim 9, wherein the mutant alpha-synuclein is mutant human alpha-synuclein A53T.

11. The method of claim 1, part e), wherein the neuronal cells are dopaminergic cells.

12. The method of claim 1, wherein the promotion of dis-aggregation, or the inhibition of aggregation, by the agent identifies the agent as a compound suitable for treating a condition associated with alpha-synuclein aggregation.

13. The method of claim 1, wherein the agent is selected from the group consisting of a chemical, a therapeutic molecule, a biomolecule, and a virus.

14. The method of claim 13, wherein the chemical is a small molecule.

15. The method of claim 13, wherein the therapeutic molecule is an antibiotic.

16. The method of claim 13, wherein the biomolecule is a polypeptide.

17. The method of claim 16, wherein the polypeptide is a peptoid.

18. The method of claim 13, wherein the biomolecule is a nucleic acid.

19. The method of claim 18, wherein the nucleic acid is DNA.

20. The method of claim 18, wherein the nucleic acid is RNA.

21. The method of claim 20, wherein the RNA is anti-sense RNA.

22. The method of claim 20, wherein the RNA is siRNA.

23. The method of claim 13, wherein the virus is a lentivirus.

24. A method of treating an individual suffering from a disease associated with alpha-synuclein aggregation, the method comprising administering to the individual a pharmaceutical composition comprising a therapeutically effective amount of an agent identified by the method of claim 1.

25. A method of treating an individual suffering from Parkinson's Disease, the method comprising administering to the individual a pharmaceutical composition comprising a therapeutically effective amount of an agent identified by the method of claim 1.

26. A system for identifying an agent, the system comprising:
a) providing a plurality of candidate agents;
b) contacting each candidate agent with a polypeptide comprising alpha-synuclein and determining whether the agent promotes alpha-synuclein dis-aggregation, or inhibits alpha-synuclein aggregation, in vitro;
c) administering an agent identified in b) to a non-mammalian organism comprising neuronal cells that over-express a polypeptide comprising alpha-synuclein and determining whether the agent promotes alpha-synuclein dis-aggregation, or inhibits alpha-synuclein aggregation, by detecting a change in the aggregation of the over-expressed alpha-synuclein;
d) administering an agent identified in c) to a transgenic mammalian organism comprising neuronal cells that over-express a polypeptide comprising alpha-synuclein in vivo and determining whether the agent promotes alpha-synuclein dis-aggregation, or inhibits alpha-synuclein aggregation, by detecting a change in the aggregation of the over-expressed alpha-synuclein;
e) optionally administering an agent identified in d) to a non-human primate genetically modified to over-express a polypeptide comprising alpha-synuclein in vivo and determining whether the agent promotes alpha-synuclein dis-aggregation, or inhibits alpha-synuclein aggregation by detecting a change in the aggregation of the over-expressed alpha-synuclein; and
f) communicating the results of b, c), d) and e), or any combination thereof, to a database.
